(19) United States
(12) Patent Application Publication
(10) Pub. No.: US 2026/0355468 A1
(43) Pub. Date: Nov. 12, 2026

(54) Catheter Placement System and Method

(71) Applicant: Bard Access Systems, Inc., Salt Lake City, UT (US)

(72) Inventors: Glade H. Howell, Draper, UT (US); Kyle G. Thornley, Farmington, UT (US); Kristin N. Rominger, South Jordan, UT (US)

(21) Appl. No.: 19/201,807

(22) Filed: May 7, 2025

(51) Int. Cl. A61M 25/06; A61M 25/00; A61M 25/09

(52) U.S. Cl. CPC A61M 25/065; A61M 25/0026; A61M 25/0097; A61M 25/09; A61M 2025/0037; A61M 2025/09133; A61M 2205/0238; A61M 2205/582; A61M 2205/584

(57) ABSTRACT

A system and method for catheter placement using a catheter placement system having a catheter, an introducer, a coupling system and a guidewire. The method involves observing a first marking on a guidewire shaft at a location between a coupling system and the catheter's distal tip to ensure the guidewire's distal tip is within the needle lumen and aligned with, or proximal to, the needle tip. The needle tip is advanced to access the vasculature, followed by advancing the guidewire. A second marking is observed indicating an atraumatic tip structure of the guidewire has been deployed. One or more additional markings are observed to indicate the start, the progress, and the completion of one or more steps in the catheter placement procedure, facilitating safe and effective catheter placement.

### BACKGROUND

In the field of catheter placement, traditional methods have often relied on manual techniques and visual estimation to guide the insertion of a catheter into a patient's vasculature. These methods typically involve the use of anatomical landmarks and tactile feedback to determine the position of the catheter and guidewire. However, such approaches can be prone to errors, leading to potential complications such as misplacement or damage to the vessel. The reliance on the practitioner's experience and skill level can also result in variability in the success and safety of the procedure.

To address these challenges, various systems have been developed to enhance the accuracy and safety of catheter placement. One approach involves the use of imaging technologies, such as ultrasound or fluoroscopy, to provide real-time visualization of the catheter and guidewire during insertion. While these imaging techniques can improve accuracy, they often require additional equipment and expertise, which may not be readily available in all clinical settings. Furthermore, the use of imaging can increase the complexity and cost of the procedure.

Another approach has been the development of guidewires with measurement indicators thereon having regularly spaced lateral lines to indicate a length of guidewire that is inserted. However, the distance markings fail to relate to position of the guidewire relative to the placement system, nor relate to the one or more steps in the placement procedure. As such, measurement markings often require at least two levels of information decoding, one to determine a distance, and a second to interpret what that distance may mean, adding complexity to the system and increasing the risk of human error.

Disclosed herein are systems and methods of catheter placement directed to address the foregoing.

### SUMMARY

In some aspects, the techniques described herein relate to a method of placing a catheter using a catheter placement system including, observing a first marking disposed on a guidewire and aligned with an indicator landmark prior to advancing the guidewire to confirm a distal tip of the guidewire is in a needle lumen coaligned with, or proximal of, a tip of a needle, the indicator landmark located at a first observation location disposed between a distal tip of the catheter and a guidewire aperture disposed on a shaft of the needle, advancing the needle into a patient to access a vasculature of the patient, and making a second observation of a proximal end of a second marking aligned with the indicator landmark to confirm transitioning of an atraumatic tip structure of the guidewire from a linear configuration to an atraumatic configuration, wherein a longitudinal length between the proximal end and a distal end of the second marking is equivalent to a length of the atraumatic tip structure.

In some aspects, the techniques described herein relate to a method, further including observing the distal end of the second marking aligned with the indicator landmark to indicate a start of a deployment of the atraumatic tip structure.

In some aspects, the techniques described herein relate to a method, further including, making a third observation of a third marking aligned with the indicator landmark to indicate a sufficient purchase length of the guidewire is disposed within the vasculature prior to removal of the needle from the vasculature.

In some aspects, the techniques described herein relate to a method, further including, making a fourth observation of a fourth marking aligned with the indicator landmark to indicate that the guidewire distal tip is at a guidewire tip target location, wherein the fourth marking extends over a second predetermined length of the guidewire to indicate a range of positions where the guidewire distal tip is within the guidewire tip target location.

In some aspects, the techniques described herein relate to a method, wherein the indicator landmark includes one of a support rod coupled with a coupling system, or an arrow disposed on an outer surface of the coupling system, the coupling system having a body defining a lumen and a slit extending from the outer surface to communicate with the body lumen, the needle slidably engageable with the body lumen and the guidewire extending through the slit to extend through the guidewire aperture of the needle shaft.

In some aspects, the techniques described herein relate to a method, further including disengaging the coupling system from the guidewire and advancing the catheter over the guidewire to place the catheter distal tip at a catheter tip target location.

In some aspects, the techniques described herein relate to a method, further including observing a fifth marking aligned with the indicator landmark to indicate the guidewire distal tip is withdrawn proximally and is proximate a point on the catheter placement system where advancement of the guidewire is prevented, the fifth marking disposed distally of the first marking and extending over a third predetermined length.

In some aspects, the techniques described herein relate to a method, further including observing a sixth marking aligned with the indicator landmark to indicate the guidewire distal tip is proximal of the point on the catheter placement system that prevents further distal advancement of the guidewire, the sixth marking disposed distally of the fifth marking and extending over a fourth predetermined length.

In some aspects, the techniques described herein relate to a method, wherein the catheter is a rapidly insertable central catheter and includes a monoluminal first section disposed distally, a catheter tube defining two or more lumen, and a transition section disposed between the monoluminal first section and the catheter tube, a catheter hub is coupled to a proximal end of the catheter tube and includes one or more extension legs, each extension leg of the one or more extension legs being in fluid communication with a lumen of the catheter tube.

In some aspects, the techniques described herein relate to a method, further including a second set of markings observable at a second observation location, a marking of the second set of markings being observable at the second observation location when one or more of the first marking, the second marking, the third marking, and the fourth marking is observable at the first observation location.

In some aspects, the techniques described herein relate to a method, wherein the second observation location is disposed between a proximal end of the catheter and a proximal end of the guidewire.

In some aspects, the techniques described herein relate to a method, further including engaging a guidewire stop coupled to the proximal end of the guidewire, with the proximal end of the catheter to prevent further distal advancement of the guidewire.

In some aspects, the techniques described herein relate to a method, wherein one or more of the first marking, the second marking, the third marking, the fourth marking, the fifth marking and the sixth marking include a colored dye, a colored paint, or a colored coating formed of a plastic or polymer, and are disposed on an outer surface of the guidewire.

In some aspects, the techniques described herein relate to a method, wherein one or more of the colored dye, the colored paint, or the colored coating extend from the outer surface of the guidewire to provide one or both of a visual marker and a tactile marker.

In some aspects, the techniques described herein relate to a method, wherein the guidewire includes a coating disposed on an outer surface of the guidewire and includes a color that contrasts with the color of the guidewire, and wherein one or more of the first marking, the second marking, the third marking, the fourth marking, the fifth marking and the sixth marking includes a portion of the coating being removed to expose a portion of the guidewire disposed therebelow.

In some aspects, the techniques described herein relate to a method, wherein one or more of the first marking, the second marking, the third marking, the fourth marking, the fifth marking and the sixth marking include an etching, engraving, notch, series of grooves, cross hatchings, knurling, or textures carved into an outer surface of the guidewire.

In some aspects, the techniques described herein relate to a catheter placement system including, a catheter extending to a distal tip, an introducer including a syringe and a needle defining a lumen, a coupling system coupled to a distal end of the syringe, and a guidewire extending through a first lumen of the catheter and through the needle lumen, the guidewire includes a first marking observable at a first observation location disposed between the catheter distal tip and the coupling system and aligned with an indicator landmark to indicate that a distal tip of the guidewire is coaligned with, or proximal of a distal tip of the needle.

In some aspects, the techniques described herein relate to a catheter placement system, further including a second marking having a longitudinal length, a distal end of the second marking being aligned with the indicator landmark at the first observation location to indicate a start of a deployment of an atraumatic tip structure, and a proximal end of the second marking being aligned with the indicator landmark to indicate the atraumatic tip structure is fully deployed, the second marking including a contrasting color relative to both the first marking and the guidewire.

In some aspects, the techniques described herein relate to a catheter placement system, further including a third marking that, when aligned with the indicator landmark, indicates a sufficient purchase length of the guidewire is disposed within a vasculature prior to removal of the needle from an insertion site, the third marking having a longitudinal length different from the longitudinal length of the second marking.

In some aspects, the techniques described herein relate to a catheter placement system, further including a fourth marking that, when aligned with the indicator landmark, indicates the guidewire distal tip is at a guidewire tip target location within the vasculature, the fourth marking having a longitudinal length that is different from the longitudinal length of both the second marking and the third marking.

In some aspects, the techniques described herein relate to a method of placing a catheter using a catheter placement system including, observing a first marking disposed on a guidewire and aligned with a first indicator landmark prior to advancing the guidewire to confirm a distal tip of the guidewire is in a needle lumen coaligned with, or proximal of, a tip of a needle, the first indicator landmark located at a first position within a first observation location disposed between a distal tip of the catheter and a guidewire aperture disposed on a shaft of the needle, advancing the needle into a patient to access a vasculature of the patient, and making a second observation of the first marking aligned with a second indicator landmark to confirm transitioning of an atraumatic tip structure of the guidewire from a linear configuration to an atraumatic configuration.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of a catheter placement system including a catheter, an introducer system, a coupling system, and a guidewire, in accordance with embodiments disclosed herein.

FIG. 1B shows a side view of the catheter placement system of FIG. 1A, in accordance with embodiments disclosed herein.

FIG. 2A shows a distal portion of an exemplary catheter for use with the catheter placement system of FIG. 1A, in accordance with embodiments disclosed herein.

FIG. 2B shows a lateral cross-section view of the catheter of FIG. 2A at first position, in accordance with embodiments disclosed herein.

FIG. 2C shows a lateral cross-section view of the catheter of FIG. 2A at a second position, in accordance with embodiments disclosed herein.

FIG. 3 shows close-up detail of a distal portion of a catheter placement system, in accordance with embodiments disclosed herein.

FIG. 4 shows a perspective view of a guidewire for use with the catheter placement system of FIG. 1A including one or more markings, in accordance with embodiments disclosed herein.

FIGS. 5A-5C show an exemplary method of advancing a guidewire of the catheter placement system, in accordance with embodiments disclosed herein.

FIGS. 6A-6C show an exemplary method of advancing a guidewire of the catheter placement system including one or more indicator landmarks, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “right,” “left,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter or system disclosed herein includes a portion of the catheter or system intended to be near or relatively nearer to a clinician when the catheter or system is used on a patient. Likewise, a “proximal length” of, for example, the catheter or system includes a length of the catheter or system intended to be near or relatively nearer to the clinician when the catheter or system is used on the patient. A “proximal end” of, for example, the catheter or system includes an end of the catheter or system intended to be near or relatively nearer to the clinician when the catheter or system is used on the patient. The proximal portion, the proximal end portion, or the proximal length of the catheter or system can include the proximal end of the catheter or system; however, the proximal portion, the proximal end portion, or the proximal length of the catheter or system need not include the proximal end of the catheter or system. That is, unless context suggests otherwise, the proximal portion, the proximal end portion, or the proximal length of the catheter or system is not necessarily a terminal portion or terminal length of the catheter or system.

With respect to “distal,” a “distal portion” or a “distal end portion” of, for example, a catheter or system disclosed herein includes a portion of the catheter or system intended to be near or relatively nearer to a patient when the catheter or system is used on a patient. Likewise, a “distal length” of, for example, the catheter or system includes a length of the catheter or system intended to be near or relatively nearer to the patient when the catheter or system is used on the patient. A “distal end” of, for example, the catheter or system includes an end of the catheter or system intended to be near or relatively nearer to the patient when the catheter or system is used on the patient. The distal portion, the distal end portion, or the distal length of the catheter or system can include the distal end of the catheter or system; however, the distal portion, the distal end portion, or the distal length of the catheter or system need not include the distal end of the catheter or system. That is, unless context suggests otherwise, the distal portion, the distal end portion, or the distal length of the catheter or system is not necessarily a terminal portion or terminal length of the catheter or system.

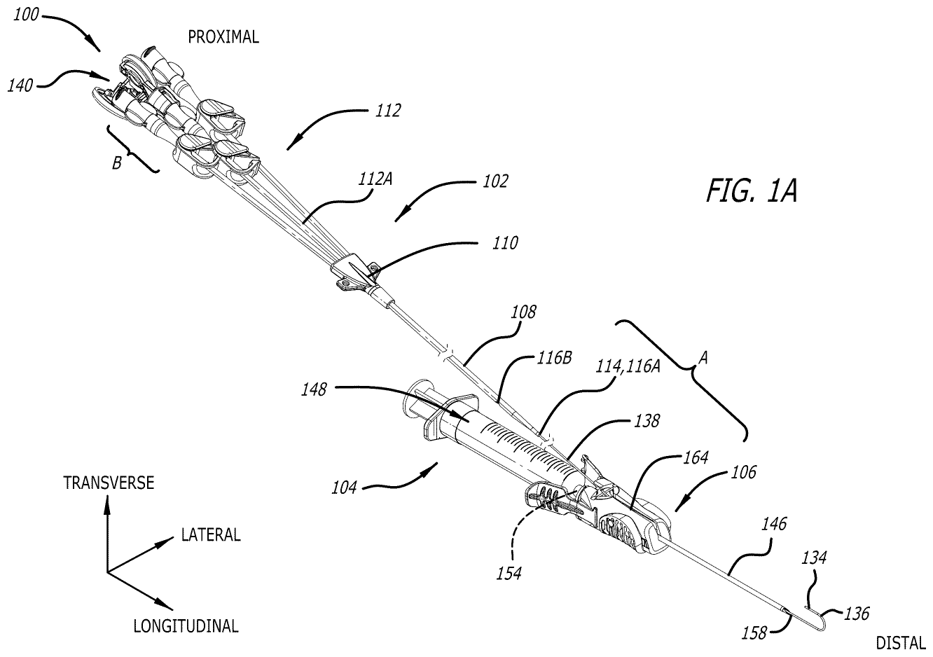

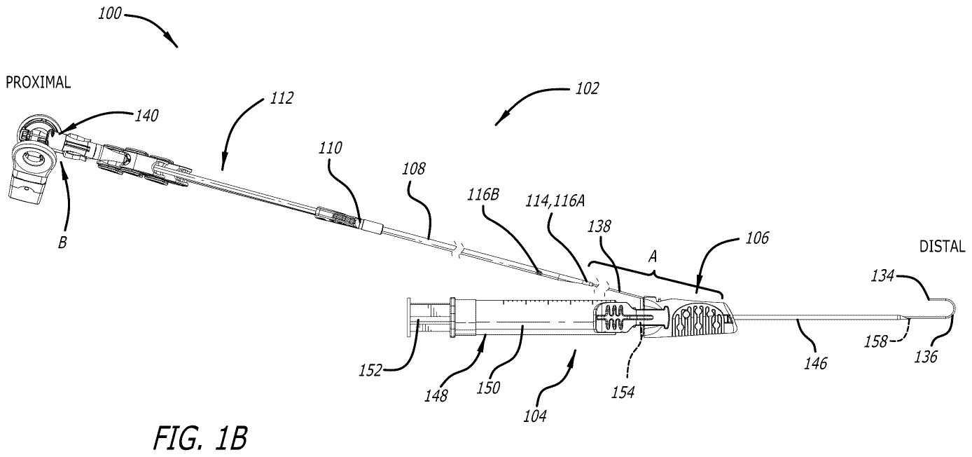

To assist in the description of embodiments described herein, as shown in FIG. 1A, a longitudinal axis extends substantially parallel to an axial length of a needle 146. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

FIGS. 1A-1B illustrate various views of a catheter placement system (“system”) 100, generally including a catheter 102, such as a Rapidly Introduced Central Catheter (“RICC”), an introducer assembly (“introducer”) 104, a coupling system 106, a needle 146, and a guidewire 138, in accordance with some embodiments. FIG. 1A shows a perspective view of the catheter placement system 100. FIG. 1B shows a side view of the catheter placement system 100. As will be appreciated the RICC 102 is exemplary and other types of catheters such as central venous catheters (CVC), peripherally inserted central catheters (PICC), midline catheters, intravenous catheters, and associated placement systems, or the like, are also contemplated to fall within the scope of the present invention.

As shown, the catheter placement system 100 includes, in some embodiments, the RICC 102, the introducer 104, and the coupling system 106 configured to couple the RICC 102 and the introducer 104 together. The RICC 102, the introducer 104, and the coupling system 106 are described in more detail herein. However, it will be appreciated that some crossover between the sections for the RICC 102, the introducer 104, and the coupling system 106 exist in view of the interrelatedness of the RICC 102, the introducer 104, and the coupling system 106 in the catheter placement system 100.

The RICC 102 includes the catheter tube 108, a catheter hub 110, and one or more extension legs 112, each extending distally from the catheter hub 110 and in fluid communication with a lumen of the catheter tube 108. The catheter tube 108 includes two or more sections including a transition section 122 and first section 118. The first section 118 having a catheter tip 114 at a distal end and defining an aperture 116A in fluid communication with a first lumen 124 of the catheter tube 108. The catheter tube 108 further includes one or more side apertures, e.g. a second aperture 116B, disposed distally and extending through a side wall of the catheter tube 108, each of the one or more side apertures communicating with a lumen of the catheter tube 108, e.g. a second lumen, as described in more detail herein.

The RICC 102 can be formed of a plastic, polymer, elastomer, rubber, silicone rubber, polytetrafluoroethylene, polypropylene, polyurethane, polyvinyl chloride, polyethylene, polyurethane, or composite, combinations thereof, or similar suitable materials. In an embodiment, the RICC 102, or one or more sections thereof, can be formed of one or more different materials, each displaying different mechanical characteristics.

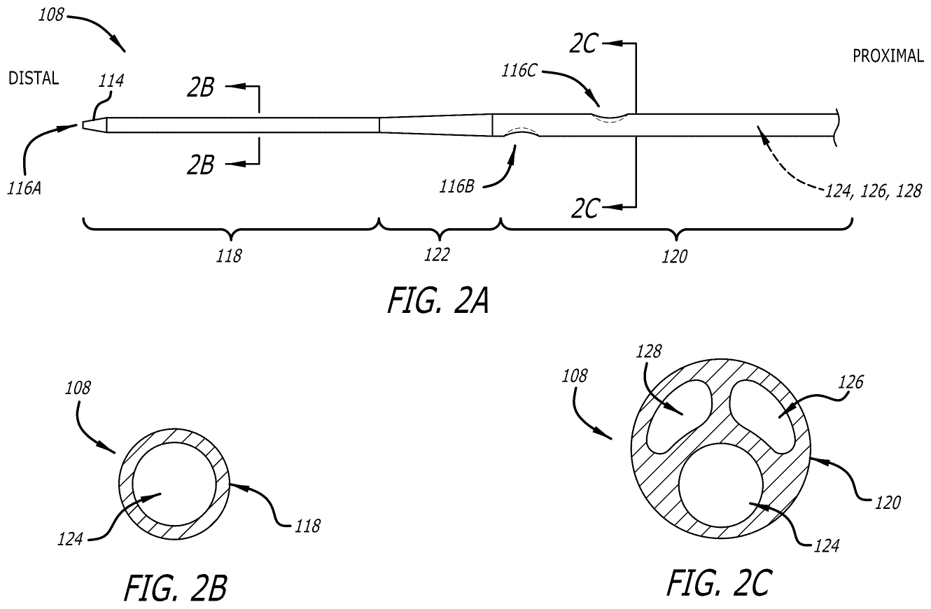

FIGS. 2A-2C show further details of a distal portion of the RICC 102 including a first section 118 disposed distally, a distal section 120 of the catheter tube 108 disposed proximally, and a transition section 122 disposed therebetween. FIG. 2A shows a side view of the distal portion of the RICC 102, FIG. 2B shows a cross-section view of the first section 118, and FIG. 2C shows a cross-section view of the distal section 120 of the catheter tube 108.

In an embodiment the catheter tube 108 defines three lumen. A first lumen 124 extends from the catheter tube 108 through the transition section 122 and through the first section 118 and communicates with a first aperture 116A disposed at a distal tip 114. A second lumen 126 and a third lumen 128 extend through the catheter tube 108 and communicate with a second aperture 116B and a third aperture 116C respectively, each disposed in a side wall of the catheter tube 108 and disposed proximally of the first section 118.

As described in more detail herein, when placing the RICC 102, the catheter placement system 100 including the first section 118 accesses a vasculature and confirms access by blood flash indication. Once access to the correct vessel is confirmed, the tapered transition section 122 is urged distally to dilate the insertion site and allow the catheter tube 108 to enter the vasculature with a distal section 120 including the second aperture 116B and the third aperture 116C disposed at a catheter tip target location. It is notable in that it makes it possible to rapidly confirm the correct vessel, dilate the insertion site and advance the catheter tube 108 through the vasculature of the patient without using the Seldinger technique.

In further reference to FIGS. 1A-1B, the catheter hub 110 is coupled to a proximal-end portion of the catheter tube 108. The catheter hub 110 includes one or more catheter-hub lumens corresponding in number to the one or more lumens of the catheter tube 108. The one or more catheter-hub lumens extend through an entirety of the catheter hub 110 from a proximal end of the catheter hub 110 to a distal end of the catheter hub 110. Each extension leg of the one or more extension legs 112 is coupled to the catheter hub 110 by a distal-end portion thereof. The one or more extension legs 112 respectively include one or more extension-leg lumens, which, in turn, correspond in number to the one-or-more catheter-tube lumens. Each extension-leg lumen of the one-or-more extension-leg lumens extends through an entirety of the extension leg from a proximal end of the extension leg to a distal end of the extension leg. Each extension leg of the one or more extension legs 112 typically includes a Luer connector coupled to the extension leg, through which Luer connector the extension leg and the extension-leg lumen thereof can be connected to another medical device.

As will be appreciated, while the RICC 102 shown in FIGS. 1A-2C is a triluminal RICC 102, embodiments described herein include RICC devices 102 having fewer or greater numbers of lumens such as monoluminal, biluminal, or multiluminal RICC's, and are contemplated to fall within the scope of the present invention. Whether the RICC 102 is monoluminal or multiluminal, the RICC 102 includes at least the first lumen 124 extending from a proximal end of a first extension leg 112A, through the catheter hub 110, the catheter tube 108, the transition section 122, and the first section 118, and communicates with the distal tip aperture 116A.

The catheter placement system 100 further includes a guidewire 138 having a guidewire shaft 130 extending between a proximal end and a distal tip 134. The guidewire 138 can include an atraumatic tip structure 136 (e.g., a coiled, partially coiled tip, U-shape, helical shape, or the like) disposed at a distal section of the guidewire shaft 130. In an embodiment, the guidewire 138 includes one or more markings 132 disposed along the shaft 130, as described in more detail herein. The guidewire 138 can be formed of a plastic, polymer, metal, alloy, composite, stainless steel, alloys including Nickel or Titanium, Nitinol, combinations thereof, or similar suitable materials.

The guidewire 138 is disposed within a lumen of the RICC 102, e.g., in a first lumen 124, and can extend from a point disposed proximally of the proximal end of the first extension leg 112A, through the first lumen 124 to a point disposed distally of the distal tip 114 of the RICC. In an embodiment, the guidewire 138 includes a stop 140 (e.g., a hub, a ball, a slug, etc.) coupled to the proximal end of the guidewire 138. The stop 140 of the guidewire 138 defines a lateral and/or transverse axis that is larger than a diameter of a proximal-end opening of the first lumen 124, e.g. the proximal end of the first extension leg 112A lumen, thereby providing a distal limit for advancing the guidewire 138 into the RICC 102.

Optionally, the guidewire 138 can be disposed in a sterile barrier (e.g., a longitudinal bag) (not shown) configured to maintain sterility of the guidewire 138 both before use (e.g., shipping and handling, storage, etc.) of the catheter placement system 100, and during use of the catheter placement system 100. During use of the catheter placement system 100, the sterile barrier is configured to provide a no-touch advancing means for advancing the guidewire 138 into a blood-vessel lumen of a patient upon establishing a needle tract thereto. Likewise, the sterile barrier is configured to provide a no-touch withdrawing means for withdrawing the guidewire 138 from the blood-vessel lumen of the patient, for example, after the catheter tube 108 has been advanced over the guidewire 138. In an embodiment, the sterile barrier is transparent to allow a clinician to observe the guidewire 138, and one or more markings 132 disposed thereon.

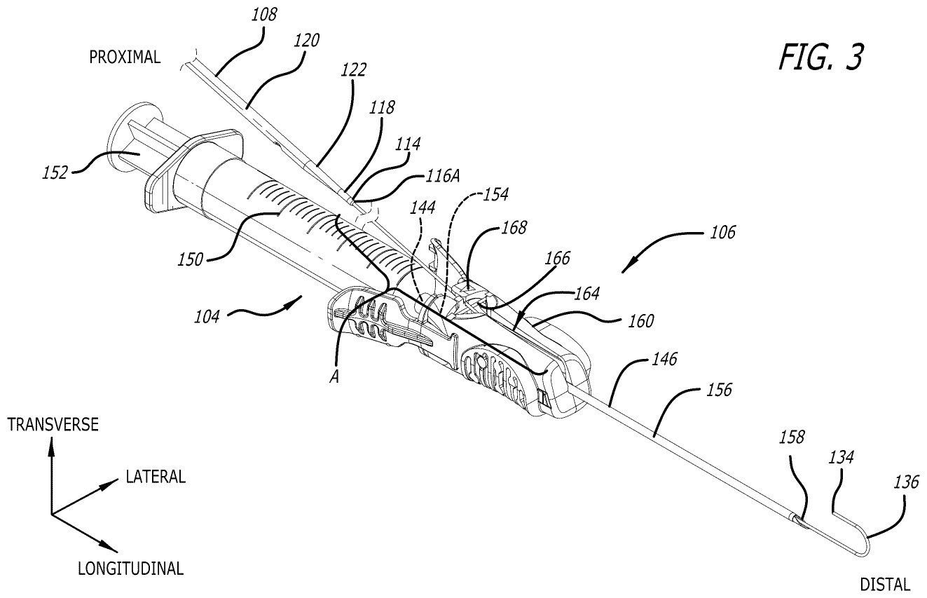

FIG. 3 shows further details of the introducer assembly 104 and coupling system 106 of the catheter placement system 100. The introducer assembly 104 includes an introducer needle 146 coupled to a syringe 148. The syringe 148 includes a syringe barrel 150 and a plunger 152. Withdrawing the plunger 152 from the syringe barrel 150 forms a vacuum within the syringe barrel 150. A lumen of the needle 146 is in fluid communication with the syringe barrel 150, as such the vacuum draws fluid proximally through the needle lumen and into the syringe barrel 150. The introducer needle 146 includes a needle hub 154 and a needle shaft 156 extending distally from the needle hub 154. The needle shaft 156 terminates at a distal end with a needle tip 158 (e.g., a beveled tip). In an embodiment, the needle hub 154 is translucent and preferably colorless for observing blood flashback from a venipuncture with the needle tip 158 (e.g., a beveled tip). A syringe tip 144 disposed at a distal end of the syringe barrel 150 is coupled to the needle hub 154 of the introducer needle 146 to provide fluid communication between the syringe barrel 150 and the lumen of the needle 146.

The coupling system 106 includes a body 160 defining a body lumen 162 extending between a proximal end and a distal end of the coupling system 106. The body 160 further includes a slit 164 extending longitudinally along the body and extending transversely between an outer surface of the body 160 and the body lumen 162. With the needle hub 154 coupled to the syringe tip 144, the needle shaft 156 slidably engages the body lumen 162 such that the needle tip 158 extends distally of the coupling system 106. The needle shaft includes a guidewire aperture disposed in a side wall of the needle and communicates with the needle lumen. A distal portion of the guidewire 138 extends from the distal tip aperture 116A of the RICC 102, through the slit 164, through the guidewire aperture of the needle shaft 156 and distally through the needle lumen to extend distally of the needle tip 158.

The syringe tip 144 and/or needle hub 154 couples with a proximal end of the body lumen 162 in an interference fit, press-fit, or snap-fit engagement. However, other mechanisms for engaging the coupling system 106 are also contemplated. Optionally, the engagement can include one or more rubber O-rings to secure the syringe tip 144 and/or needle hub 154 with a proximal end of the body lumen 162 and provide a fluid-tight seal therebetween. The coupling system 106 further includes a seal disposed within the body lumen 162 and/or slit 164 and configured to encircle the guidewire aperture of the needle shaft to mitigate fluid leakage to or from the junction between the guidewire 138 and the needle 146.

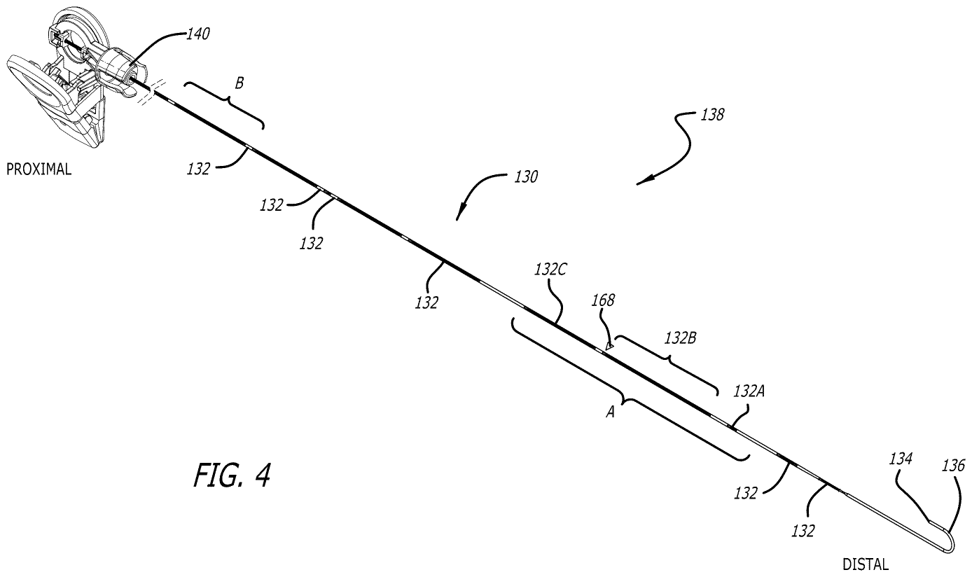

FIG. 4 shows a perspective view of the guidewire 138 and stop 140 with the rest of the RICC system removed for ease of illustration. In an embodiment, the guidewire 138 includes one or more markings 132 disposed on a shaft 130 thereof. In embodiments, one or more of the markings 132 can include a colored dye, paint, or plastic coating disposed on an outer surface of the guidewire 138 and contrasts with the color of the rest of the guidewire 138 so that they are easily observed by visual inspection. Optionally the colored marking can extend from the surface of the guidewire 138 to provide both a visual and a tactile indication. In an embodiment, the guidewire 138 is coated with a coating having a contrasting color relative to the guidewire disposed there below. One or more of the markings 132 can include portions of the coating that is removed to show the color of the guidewire 138 disposed therebelow.

In an embodiment, one or more of the markings 132 include an etching, engraving, notch, or similar structure or pattern carved into the surface of the guidewire by engraving or similar mechanical means, acid etching or similar chemical means, or laser ablation or similar means. Advantageously, the removal of the portion of coating and/or guidewire to form the one or more of the markings 132 can provide a recess or texture in the outer surface of the guidewire 138 to provide both a visual and a tactile indication. One or more of the markings 132 can include different patterns, for example a series of grooves extending parallel or at an angle relative to a longitudinal axis of the guidewire 138, a series of coils or helices extending about the longitudinal axis, cross hatchings, knurling, or randomized textures.

In an embodiment, the color and/or texture of the marking can vary, along a longitudinal length of the marking, between one or more colors and/or one or more textures. For example, a color of the marking can fade from a first color to a second color, and/or a texture can gradually change from a first pattern or texture (rough) to a second pattern or texture (relatively smooth), to indicate a degree of completion of the process. These and other combinations of marking variation within a marking are contemplated to fall within the scope of the present invention.

In an embodiment, one or more of the markings 132 can extend annularly about the longitudinal axis of the guidewire 138, or can extend partially around the longitudinal axis of the guidewire 138. In an embodiment, one or more of the markings 132 can extend over different longitudinal lengths of the guidewire shaft 130. In an embodiment, one or more of the markings 132 can include materials that allow the markings 132 to be observed under different imaging modalities. For example, the markings 132 can include radiopaque materials or materials having different mechanical or chemical properties that allow the markings to be observed under fluoroscopy, ultrasound, electromagnetic (EM), positron emission tomography-computed tomography (PET-CT) imaging, magnetic resonance imaging (MRI), or similar medical imaging modalities.

As shown, each marking 132 can be disposed at a predetermined location along the guidewire shaft 130 to facilitate deployment of the catheter placement system 100. It is important to note that the various steps required in deploying the catheter placement system 100 require confirmation of actions that are not directly observable. For example, during the first advancement stage of the guidewire 138, the clinician requires to advance the guidewire distal tip 134 sufficiently such that the atraumatic tip structure 136 extends from the needle tip 158 and resumes the elastically undeformed atraumatic shape (e.g., a coiled, partially coiled tip, U-shape, helical shape, or the like). However, the start and end of the deployment process cannot be directly observable since this occurs intravascularly. Using medical imaging systems to observe the guidewire 138 can be cumbersome, expensive, and may not provide the necessary resolution to observe the guidewire at all. Further, various steps in the placement procedure require quick feedback given the speed that the catheter placement system 100 is intended to be used. As such, any delay in feedback, e.g., incurred while using a medical imaging system compared to direct observation, may compromise the procedure.

When deploying the atraumatic tip structure 136, a clinician is required to advance the guidewire 138 sufficiently far that the entire atraumatic tip structure 136 is distal of the needle tip 158 to allow the atraumatic tip structure 136 to resume the atraumatic shape. If the atraumatic tip structure 136 is only partially deployed from the needle tip 158 then the tip of the guidewire 138 may extend towards, and may accidently puncture, the vessel wall. Similarly, if the guidewire distal tip 134 is extended too far and/or too fast the atraumatic tip structure 136 may not have time to resume the undeformed atraumatic shape. As such, the tip of the guidewire may be urged through the vessel wall. As will be appreciated, if the atraumatic tip structure 136 is deformed to the linear shape within the needle lumen during transport and storage, the atraumatic tip structure 136 may be held in this linear position for an extended period of time and may require some time, e.g. a few seconds to a minute, to resume the undeformed atraumatic shape. In addition, the atraumatic tip structure 136 may be relative cold depending on the temperature external to the patient and so may be relatively stiff, preventing the atraumatic tip structure 136 from resuming the undeformed shape as fast, relative to when it is warm, e.g. body temperature.

Advantageously, the catheter placement system 100 includes one or more markings 132 disposed on the shaft of the guidewire 138 and configured to indicate one or more steps in the placement procedure. Exemplary steps can include but are not limited to, indicating when a tip of the guidewire is, proximal, co-aligned with, or distal of the needle tip 158, when the atraumatic tip structure 136 is being deployed or is fully deployed, when there is sufficient purchase length of the guidewire in the vessel such that the needle 146 can be removed, or when the guidewire distal tip 134 is at a target location, as described in more detail herein.

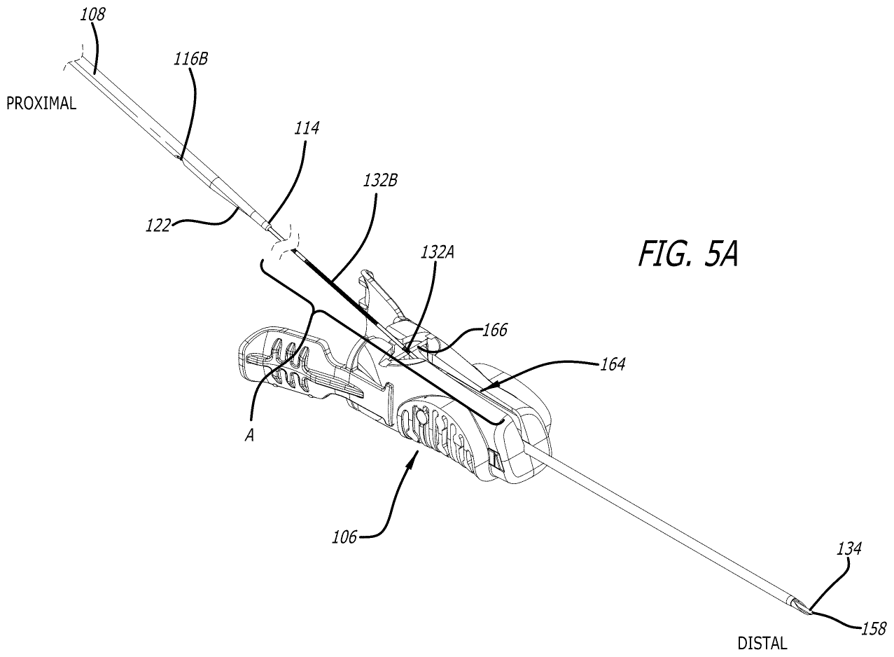

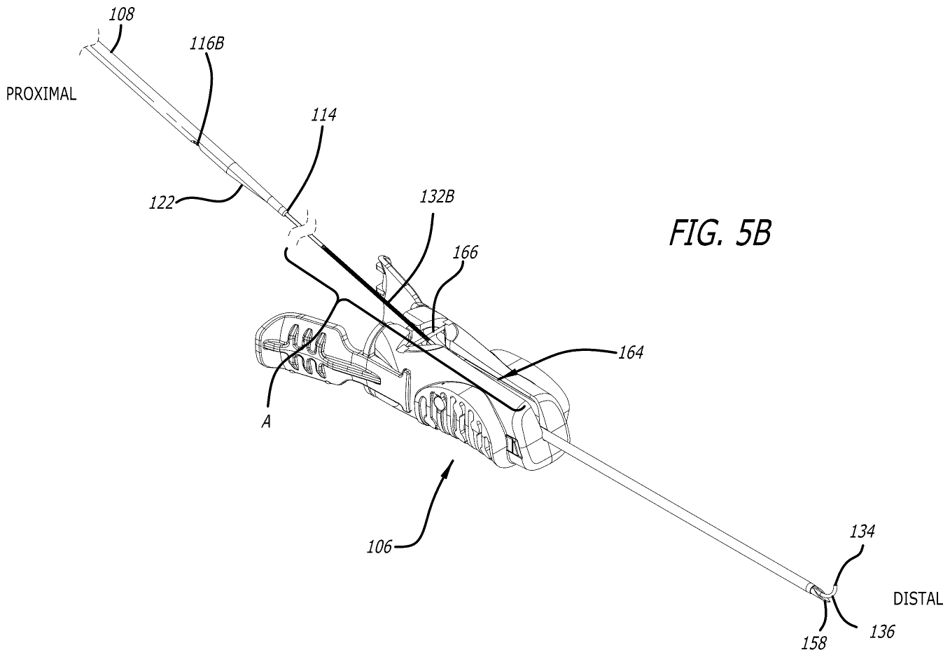

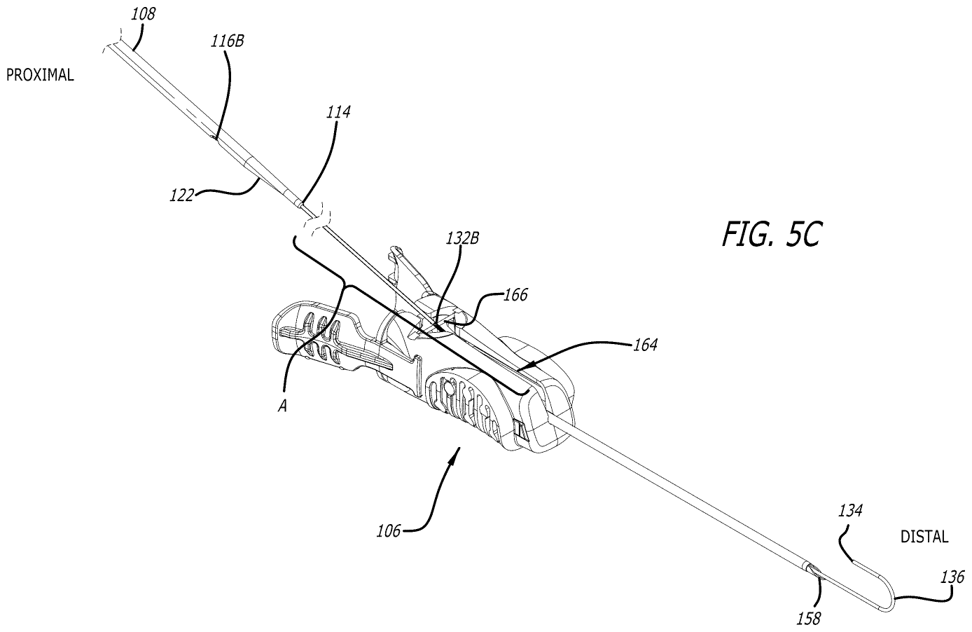

By way of an illustrative example, as shown in FIGS. 4-5C, a first marking 132A can be disposed on the guidewire shaft 130 and a predetermined distance from the guidewire distal tip 134. As such, as shown in FIG. 5A, when the guidewire distal tip 134 is aligned with the needle tip 158, the first marking 132A can be observed by the clinician, for example at an indicator landmark at observation location “A” disposed between the RICC tip 114 and a guidewire aperture of the needle 146. In an embodiment, the catheter system 100 includes an indicator landmark to indicate the exact point in the guidewire advancement process. In an embodiment, the indicator landmark can be a support rod 166 configured to support a portion the guidewire 138 extending from the slit 164 and directly observable by a clinician. In an embodiment, the indicator landmark includes a symbol, icon, arrow, or similar visual indicator 168 disposed on an outer surface of the coupling system 106 and adjacent the slit 164 where a portion of the guidewire 138 extending into the slit 164 is directedly observable. (FIG. 3). Advantageously, one or more markings 132, or portions thereof, can be observed proximally and/or distally of the indicator landmark, e.g., support rod 166 or symbol 168, to allow the clinician to observe which steps have been performed, and which steps have yet to be performed, along with an indication of relative time since these steps have been, or will be, performed.

In an embodiment, the first marking 132A can be a lateral line to indicate a precise position. Further the first marking 132A can include a color, pattern, symbol or alphanumeric symbol to encode information as to what the first marking 132A corresponds to. In an embodiment, a second marking 132B is disposed on the guidewire shaft 130 to indicate that the atraumatic tip structure 136 is being deployed. The second marking 132B can indicate a start of the deployment and is co-aligned with or disposed proximally of the first marking 132A. A third marking 132C can be disposed proximally of the second marking 132B and indicate that the atraumatic tip structure has been fully deployed.

In an embodiment, as shown in FIGS. 4-5C the second marking 132B extends longitudinally along the guidewire shaft 130 such that a distal end of the second marking 132B indicates a start of the deployment and a proximal end of the second marking 132B indicates the atraumatic tip structure 136 is fully deployed. Worded differently, the longitudinal length of the second marking 132B is equivalent to, or longer than, a length of a portion of the guidewire 138 that forms the atraumatic tip structure 136.

As shown in FIG. 5A, a first marking 132A is aligned with an indicator landmark, rod 166 to indicate the guidewire distal tip 134 is coaligned with the needle tip 158. As shown in FIG. 5B, the guidewire 138 is advanced distally through the RICC 102 and catheter placement system 100 until a distal end of the second marking 132B is aligned with the indicator landmark to indicate the guidewire distal tip is advanced distally of the needle tip 134 and the distal end of the atraumatic tip structure 136 is co-aligned with, or distal of, the needle tip 158. As shown in FIG. 5C, a proximal end of the second marking 132B is aligned with the indicator landmark indicates the entire atraumatic tip structure 136 is distal of the needle tip 158 and is fully deployed.

Advantageously, a clinician can observe the observation location “A” to observe one or more markings 132, together with the longitudinal position of the one or more markings 132 relative to the indicator landmark and can instantly determine what stage is in process, how far the current stage has progressed, along with which stages have already been completed, distal of the indicator landmark, and which stages have yet to be completed, proximal of the indicator landmark.

Advantageously, the distinctive color or pattern of the second marking 132B can indicate to a clinician that the process of deploying the atraumatic tip structure 136 is in process. This allows the clinician to easily keep track of the insertion process compared to separate lateral linear markings indicating the start and finish of the deployment process, which may be less easily observed. For example, a clinician may miss seeing a marking and advance to the next stage of the placement process prematurely risking trauma to the vessel. Alternatively, the marking indicating the start of the process may disappear from view into the coupling system body lumen 162 before the next marking indicating the end of the process has emerged from the distal tip aperture 116A. As such, the clinician may lose track of which stage in the process they are at and advance to the next stage of the placement process prematurely risking trauma to the vessel.

It is important to note that the one or more markings 132 are disposed at predetermined positions along the length of the guidewire 138 and extend over predetermined longitudinal lengths that may be the same or different lengths. These markings 132 at predetermined positions and lengths are specific to the catheter placement system 100 and the associated steps in the placement of the catheter 102 that the guidewire 138 is being used with. Advantageously, the configuration of the one or more markings 132 being specific to the RICC system and procedure ensures accuracy in the deployment steps, simplifies the deployment process for the clinician, and leads to faster and more accurate placement of the RICC 102, while mitigating human error. The one or more markings 132 can be observed and instantly indicate not only the current step in the placement process but also the start, the progress, or completion of that step. Differently, measurement markings which are linear and placed at regular intervals between consecutive markings, adds time and confusion to the placement process. The plurality of regular markings can be difficult to read and keep track of, especially when being advanced rapidly past an observation location, e.g. location “A.” Further, the clinician is required to perform an extra step of translating a measured distance to a specific procedure step, risking increased human error and failure of the procedure.

In an embodiment, a first set of one or more markings 132 can be disposed towards a distal end of the guidewire shaft 130. In an embodiment, a second set of one or more markings 132 can be disposed towards a proximal end of the guidewire shaft 130 and can be observed, as described herein, at a location “B” between a guidewire stop 140 and a proximal end of the first extension leg 112A. To note, FIGS. 1A-1B show the guidewire stop 140 in the distal most position for ease of illustration. However, as will be appreciated, when the guidewire distal tip 134 is positioned relative to the needle tip 158 as shown in FIGS. 1A-1B, the guidewire stop 140 will instead be positioned proximally allowing a clinician to observe a portion of the guidewire shaft 130 between the guidewire stop 140 and the proximal end of the first extension leg 112A. Each marking 132 of the second set of markings denotes a step in the catheter placement process. The point at which the marking disappears into the lumen of the first extension leg 112A indicates the exact point in the placement process. Worded differently, the indicator landmark for the location “B” is the proximal opening of the first lumen 124. Advantageously, at location “B”, the clinician can observe all of the remaining steps of the placement process, i.e. those markings that are still proximal of the lumen of the first extension leg 112A, and can keep track of which stage of the procedure is in process and which steps are still to be performed.

In an embodiment, additional markings 132 can be disposed distally of the first marking 132A to indicate that the guidewire 138 has been retracted from the position where the guidewire distal tip 134 and needle tip 158 are co-aligned. These markings 132 can indicate if the guidewire distal tip 134 is still in the needle lumen and as such can still be deployed successfully. These markings 132 can also indicate if the guidewire distal tip 134, or other points on the guidewire 138, are nearing a point, or is past a point that might cause the system 100 to malfunction. For example, if the guidewire distal tip 134 is nearing the guidewire aperture of the needle a marking 132, e.g. colored yellow, can indicate that the guidewire distal tip 134 is close to a point that will prevent the system from deploying correctly, and that caution should be used to prevent further withdrawal. As described above the marking can extend over a given longitudinal length of the guidewire shaft 130 to indicate a range of guidewire positions fall within this warning zone. Further, when the guidewire distal tip 134 is withdrawn proximally of the needle aperture a different marking, e.g. colored red, can indicate a malfunction of the system 100. For example, if the guidewire distal tip 134 is withdrawn from the needle lumen the atraumatic tip structure 136 may be accidentally deployed preventing the guidewire distal tip 134 from being reinserted into the needle lumen without compromising the sterility of the system 100, and the system 100 must be disposed of. Other points on the guidewire 138 that may cause malfunction of the system 100 include a tapered section that when pulled through the seal in the coupling system 106 may compromising the fluid tight seal. As such, one or more markings 132 can indicate when the tapered section is close to the seal, and/or aligned with the seal.

In an exemplary method of use, a catheter placement system 100 is provided as described herein, e.g. FIG. 1A. Prior to deployment the syringe tip 144 and/or needle hub 157 is engaged with the coupling system 106. The introducer needle 146 extends through the coupling system body lumen 162 with a needle tip 158 extending distally from the coupling system 106. A guidewire 138 extends through the first lumen 124 of the RICC 102, a distal end of the guidewire 138 extends from the distal tip aperture 116A, through the coupling system body 160, and through the needle lumen. A distal tip of the guidewire 138 can be aligned with a needle tip 158 or can be disposed proximally of the needle tip 158. The atraumatic tip structure 136 can be elastically deformed to a linear configuration when disposed within the needle lumen.

As will be appreciated the guidewire distal tip 134, disposed within the needle lumen, cannot be directly observed. One or more markings 132, e.g. a first marking 132A, is disposed on the guidewire shaft at a predetermined position and can be observed at location “A” and/or location “B” to confirm that the guidewire distal tip 134 and needle tip 158 are coaligned and the system 100 is ready for deployment.

The catheter placement system 100 is manipulated by the clinician to pierce a skin surface of a patient, forming an insertion site. The needle tip 158 is advanced percutaneously until the needle tip 158 accesses a vessel of the patient. The plunger 152 can be partially withdrawn to create a vacuum in the syringe barrel 150 that draws a fluid through the needle lumen and into the syringe barrel 150. A color and pulsatile flow of the fluid can be observed through the transparent walls of the syringe barrel 150 to confirm the correct vessel has been accessed, e.g. an artery or vein. If the incorrect vessel is accessed, the needle tip 158 can be withdrawn and pressure applied to the insertion site to close the site without vascular surgery since the gauge of the needle 146 is relative thin.

Once the correct vessel has been confirmed, the clinician can advance the guidewire 138 such that the distal tip of the guidewire extends distally from the needle tip 158. One or more markings 132 can be observed, as described herein, to indicated when the guidewire distal tip 134 is distal of the needle tip 158, when the atraumatic tip structure 136 is being deployed, and when the atraumatic tip structure 136 is fully deployed (FIGS. 5A-5C). The clinician can then advance the guidewire into the vasculature and the atraumatic tip structure 136 can mitigate damaged to the vessel walls.

One or more markings 132 can indicate to the clinician that the guidewire has been advanced sufficiently to prevent the guidewire being accidently withdrawn from the vessel as the needle 146 is removed, termed “purchase length”. As described herein the purchase length marking can extend over a longitudinal length to indicate a range of guidewire positions where the purchase length is sufficient. With the guidewire purchase length markings 132 being observable, the clinician can withdraw the introducer assembly 104 from the coupling system 106. To note, the needle shaft 156 includes a slit or tear line extending from the guidewire aperture of the needle to a distal tip to allow a portion of the guidewire 138 that is extending through the guidewire aperture of the needle 146 to slid out from and disengage the needle 146 as the introducer assembly 104, including the syringe 148 and needle 146 is withdrawn and disengages from the coupling system 106.

The guidewire 138 can be advanced until a guidewire distal tip 134 is disposed at a guidewire tip target location. One or more markings 132 can be observable to indicate that the guidewire distal tip 134 is at the guidewire tip target location. As described herein, the target location marking 132 can extend over a predetermined length of the guidewire 138 to indicate a range of positions when the guidewire distal tip 134 is at the target location. Further, a proximal end of the target location marking 132 can indicated when the guidewire distal tip 134 has been advanced too far and risks misplacement of the catheter or may cause trauma to the patient. For example, when placing a catheter in a lower ⅓ of the superior vena cava (“SVC”) of a heart of the patient, placing the guidewire distal tip 134, and therefore the catheter distal tip 114 short of the target location results in the medication being ineffective. Placing the guidewire distal tip 134 too far can cause arrhythmia, or other complications.

In an embodiment, the step of withdrawing the needle from the coupling system 106 can be performed after the guidewire distal tip 134 has been advanced to the target location. As will be appreciated the one or more markings 132 can be configured to indicate different steps in the placement procedure, and/or in which order these steps are to be performed.

With the guidewire distal tip 134 at the target location, the coupling system 106 can be detached from the guidewire 138 but urging a portion of the guidewire 138 through the slit 164 of the coupling system 106. The RICC 102 can then be advanced over the guidewire 138, advancing a RICC tip 114 through the insertion site. As the transition section 122 is advanced through the insertion site, the transition section 122 dilates the insertion site to facilitate advancement of the catheter tube 108 over the guidewire until a distal section 120 of the catheter tube 108 is disposed at a catheter tip target location. As will be appreciated catheter tip target location may disposed proximally of the target location of the guidewire distal tip 134 since the apertures 116B, 116C are proximal of the catheter distal tip 114. The guidewire 138 can then be withdrawn proximally from the first lumen 124 and the catheter hub 110 secured to the skin of the patient.

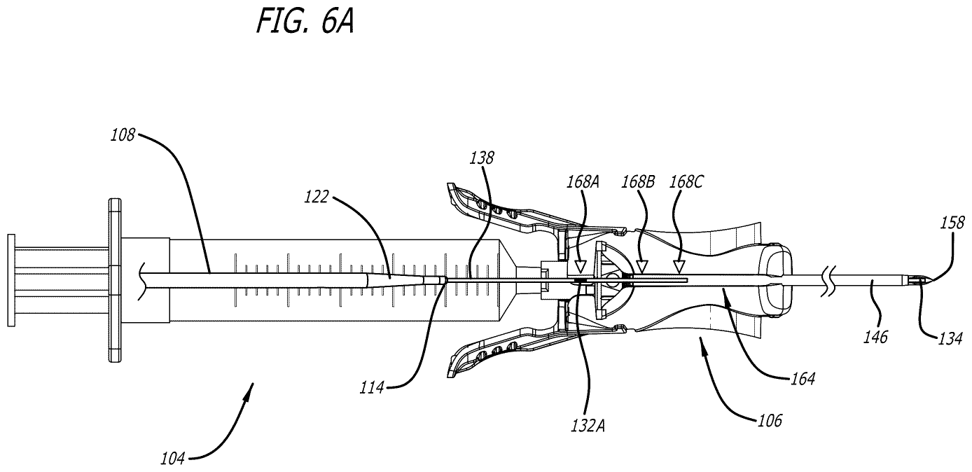

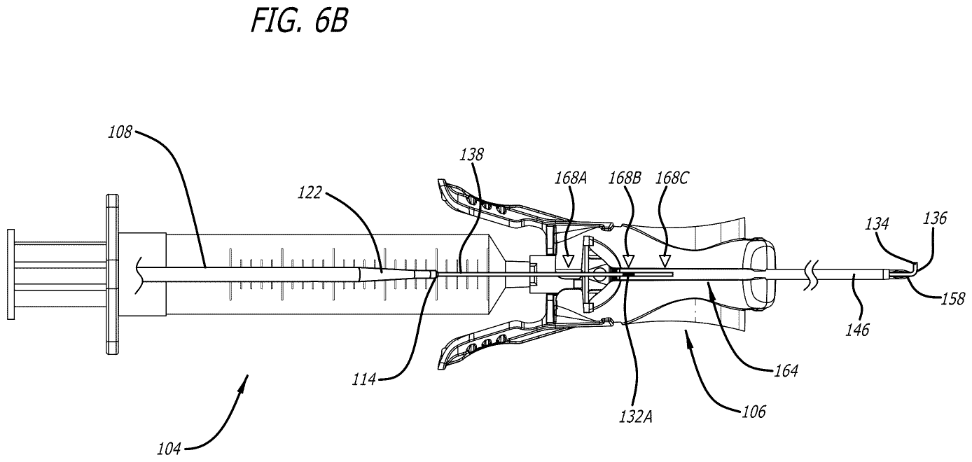

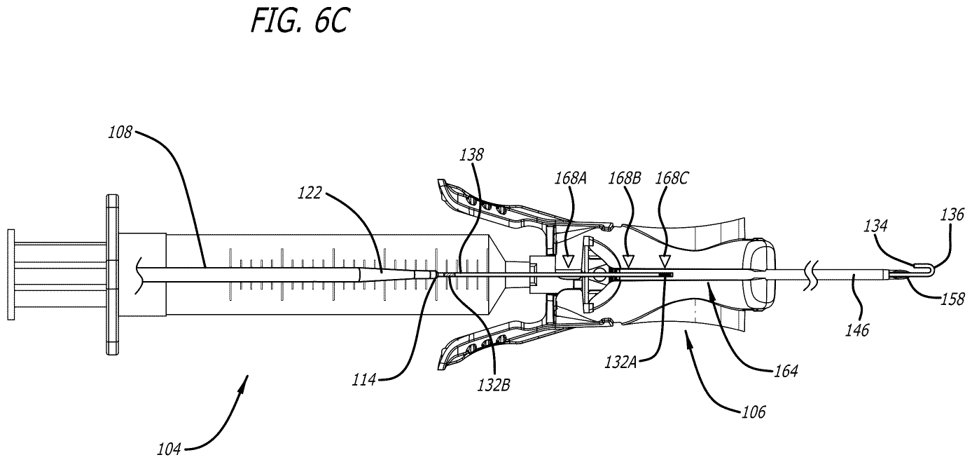

In an embodiment, as shown in FIGS. 6A-6C, the system 100 includes a guidewire 138 having one or more markings, e.g. a first marking 132A, as described herein. Further, the system 100 includes one or more indicator landmarks, as described herein. As shown, the coupling system 106 includes three indicator landmarks disposed along the slit 164 such as a first indicator landmark 168A, a second indicator landmark 168B, and a third indicator landmark 168C. However, as will be appreciated, the one or more indicator landmarks can be placed at other positions on the system 100. As the guidewire 138 is advanced or withdrawn, the first marking 132A aligns an indicator landmark of the one or more indicator landmarks to indicate a step in the placement process.

In an exemplary method of use, as shown in FIG. 6A, the guidewire 138 is disposed at a first position where a guidewire distal tip 134 is aligned with a needle tip 158. The first marking 132A is aligned with the first indicator landmark 168A to indicate to the clinician that the guidewire distal tip 134 and needle tip 158 are aligned since these structures will not be directly observable. As shown in FIG. 6B, the clinician can advance the guidewire 138 until the first marking 132AA aligns with the second indicator landmark 168B to indicate that the atraumatic tip 136 is starting to be deployed. As shown in FIG. 6C, the clinician can advance the guidewire 138 until the first marking 132AA aligns with the third indicator landmark 168C to indicate that the atraumatic tip 136 is fully deployed.

In an embodiment, the first marking 132A includes a first color or pattern to indicate that these steps are part of a first stage of the catheter placement, such as the deployment of the atraumatic tip 136 stage. As shown in FIG. 6C, the guidewire 138 further includes a second marking 132B that includes a second color or pattern. When the second marking 132B is aligned with an indicator landmark of the one or more indicator landmarks indicates the progression of one or more steps of a second stage of the catheter placement process. Additional markings 132 can indicate additional steps and/or stages as disclosed herein. As will be appreciated, the markings 132 can extend over different longitudinal lengths to indicate different step progressions. For example, where the progression of a step requires advancing the guidewire 138 over a given longitudinal distance, the marking associated with that step can extend over a similar longitudinal length of the guidewire 138. These and other configurations of one or more markings 132 and one or more indicator landmarks, e.g. indicator landmark 168, disposed on the system 100 are contemplated to fall within the scope of the present invention.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A method of placing a catheter using a catheter placement system, comprising:
observing a first marking disposed on a guidewire and aligned with an indicator landmark prior to advancing the guidewire to confirm a distal tip of the guidewire is in a needle lumen coaligned with, or proximal of, a tip of a needle, the indicator landmark located at a first observation location disposed between a distal tip of the catheter and a guidewire aperture disposed on a shaft of the needle;
advancing the needle into a patient to access a vasculature of the patient; and
making a second observation of a proximal end of a second marking aligned with the indicator landmark to confirm transitioning of an atraumatic tip structure of the guidewire from a linear configuration to an atraumatic configuration, wherein a longitudinal length between the proximal end and a distal end of the second marking is equivalent to a length of the atraumatic tip structure.

2. The method according to claim 1, further including observing the distal end of the second marking aligned with the indicator landmark to indicate a start of a deployment of the atraumatic tip structure.

3. The method according to claim 1, further comprising:
making a third observation of a third marking aligned with the indicator landmark to indicate a sufficient purchase length of the guidewire is disposed within the vasculature prior to removal of the needle from the vasculature.

4. The method according to claim 3, further comprising:
making a fourth observation of a fourth marking aligned with the indicator landmark to indicate that the guidewire distal tip is at a guidewire tip target location, wherein the fourth marking extends over a second predetermined length of the guidewire to indicate a range of positions where the guidewire distal tip is within the guidewire tip target location.

5. The method according to claim 1, wherein the indicator landmark includes one of a support rod coupled with a coupling system, or an arrow disposed on an outer surface of the coupling system, the coupling system having a body defining a lumen and a slit extending from the outer surface to communicate with the body lumen, the needle slidably engageable with the body lumen and the guidewire extending through the slit to extend through the guidewire aperture of the needle shaft.

6. The method according to claim 5, further including disengaging the coupling system from the guidewire and advancing the catheter over the guidewire to place the catheter distal tip at a catheter tip target location.

7. The method according to claim 4, further including observing a fifth marking aligned with the indicator landmark to indicate the guidewire distal tip is withdrawn proximally and is proximate a point on the catheter placement system where advancement of the guidewire is prevented, the fifth marking disposed distally of the first marking and extending over a third predetermined length.

8. The method according to claim 7, further including observing a sixth marking aligned with the indicator landmark to indicate the guidewire distal tip is proximal of the point on the catheter placement system that prevents further distal advancement of the guidewire, the sixth marking disposed distally of the fifth marking and extending over a fourth predetermined length.

9. The method according to claim 1, wherein the catheter is a rapidly insertable central catheter and includes a monoluminal first section disposed distally, a catheter tube defining two or more lumen, and a transition section disposed between the monoluminal first section and the catheter tube, a catheter hub is coupled to a proximal end of the catheter tube and includes one or more extension legs, each extension leg of the one or more extension legs being in fluid communication with a lumen of the catheter tube.

10. The method according to claim 4, further including a second set of markings observable at a second observation location, a marking of the second set of markings being observable at the second observation location when one or more of the first marking, the second marking, the third marking, and the fourth marking is observable at the first observation location.

11. The method according to claim 10, wherein the second observation location is disposed between a proximal end of the catheter and a proximal end of the guidewire.

12. The method according to claim 11, further including engaging a guidewire stop coupled to the proximal end of the guidewire, with the proximal end of the catheter to prevent further distal advancement of the guidewire.

13. The method according to claim 8, wherein one or more of the first marking, the second marking, the third marking, the fourth marking, the fifth marking and the sixth marking include a colored dye, a colored paint, or a colored coating formed of a plastic or polymer, and are disposed on an outer surface of the guidewire.

14. The method according to claim 13, wherein one or more of the colored dye, the colored paint, or the colored coating extend from the outer surface of the guidewire to provide one or both of a visual marker and a tactile marker.

15. The method according to claim 8, wherein the guidewire includes a coating disposed on an outer surface of the guidewire and includes a color that contrasts with the color of the guidewire, and wherein one or more of the first marking, the second marking, the third marking, the fourth marking, the fifth marking and the sixth marking includes a portion of the coating being removed to expose a portion of the guidewire disposed therebelow.

16. The method according to claim 8, wherein one or more of the first marking, the second marking, the third marking, the fourth marking, the fifth marking and the sixth marking include an etching, engraving, notch, series of grooves, cross hatchings, knurling, or textures carved into an outer surface of the guidewire.

17. A catheter placement system, comprising:
a catheter extending to a distal tip;
an introducer including a syringe and a needle defining a lumen;
a coupling system coupled to a distal end of the syringe; and
a guidewire extending through a first lumen of the catheter and through the needle lumen, the guidewire includes a first marking observable at a first observation location disposed between the catheter distal tip and the coupling system and aligned with an indicator landmark to indicate that a distal tip of the guidewire is coaligned with, or proximal of a distal tip of the needle.

18. The catheter placement system according to claim 17, further including a second marking having a longitudinal length, a distal end of the second marking being aligned with the indicator landmark at the first observation location to indicate a start of a deployment of an atraumatic tip structure, and a proximal end of the second marking being aligned with the indicator landmark to indicate the atraumatic tip structure is fully deployed, the second marking including a contrasting color relative to both the first marking and the guidewire.

19. The catheter placement system according to claim 18, further including a third marking that, when aligned with the indicator landmark, indicates a sufficient purchase length of the guidewire is disposed within a vasculature prior to removal of the needle from an insertion site, the third marking having a longitudinal length different from the longitudinal length of the second marking.

20. The catheter placement system according to claim 19, further including a fourth marking that, when aligned with the indicator landmark, indicates the guidewire distal tip is at a guidewire tip target location within the vasculature, the fourth marking having a longitudinal length that is different from the longitudinal length of both the second marking and the third marking.

21. A method of placing a catheter using a catheter placement system, comprising:
observing a first marking disposed on a guidewire and aligned with a first indicator landmark prior to advancing the guidewire to confirm a distal tip of the guidewire is in a needle lumen coaligned with, or proximal of, a tip of a needle, the first indicator landmark located at a first position within a first observation location disposed between a distal tip of the catheter and a guidewire aperture disposed on a shaft of the needle;
advancing the needle into a patient to access a vasculature of the patient; and
making a second observation of the first marking aligned with a second indicator landmark to confirm transitioning of an atraumatic tip structure of the guidewire from a linear configuration to an atraumatic configuration.
